(19) United States
(12) Patent Application Publication
(10) Pub. No.: US 2023/0407016 A1
(43) Pub. Date: Dec. 21, 2023

(54) POLYSACCHARIDE-INCLUDING LIQUID MATERIAL AND ITS MANUFACTURING METHOD

(71) Applicants: RENSSELAER POLYTECHNIC INSTITUTE, Troy, NY (US); OTSUKA PHARMACEUTICAL FACTORY, INC., Naruto, Tokushim (JP); OTSUKA CHEMECAL CO., LTD., Chuo-Ku, Osaka (JP)

(72) Inventor: Pengyu XU, Tokushima City (JP)

(21) Appl. No.: 18/027,712

(22) PCT Filed: Sep. 21, 2021
(86) PCT No.: PCT/IB2021/000638

§ 371 Date: Mar. 22, 2023

Related U.S. Application Data
(60) Provisional application No. 63/081,457, filed on Sep. 22, 2020.

(51) Int. Cl. C08J 3/28; C08J 3/03; A23L 33/125; A61K 8/73

(52) U.S. Cl. CPC C08J 3/28; C08J 3/03; A23L 33/125; A61K 8/735; A61K 2800/82; C08J 2305/06; C08J 2305/10; A61K 2800/81; C08J 2305/08

(57) ABSTRACT

A liquid material including a polysaccharide is manufactured by creating a mixture of polysaccharides and an aqueous solvent or an aqueous cosolvent with organic solvents at a temperature at which gelation does not occur. The mixture is also irradiated at this lower temperature, such as with ultrasound waves in combination with stirring and/or bubbling of inert gases. to apply ultrasound to a mixture of. Compared with conventional reactions with low concentration solutions, the product yield and advantageous mechanical properties are significantly increased. Depolymerization (decomposition, disassembly) of polysaccharide in the liquid material does not occur to any substantial degree, and the molecular structure of the polysaccharide is maintained. Further, the liquid material does not gel even when the temperature is raised, and thus, its fluidity is maintained, despite polysaccharides being maintained at a high concentration. The liquid material is applicable to myriad industries, including pharmaceuticals and health foods.

### CROSS REFERENCE TO RELATED APPLICATION(S)

This application is a national stage filing of International Application No. PCT/IB2021/000638, filed Sep. 21, 2021, which claims the benefit of U.S. Provisional Application No. 63/081,457, filed Sep. 22, 2020, which is incorporated by reference as if disclosed herein in its entirety.

### TECHNICAL FIELD

The present invention relates to a polysaccharide-including liquid material and its manufacturing method.

### BACKGROUND

Polysaccharides are widely used in foods and food processing, paper and paper processing, fiber processing, paint inks, construction, pharmaceuticals, nutraceuticals, cosmetics, and household products, etc. In some cases, chemical modification is required to achieve improved physicochemical and functional properties.

In order to chemically modify polysaccharides, reaction in a solution state is usually applied, and the reaction is typically performed in a very dilute solution of 1% (w/v) or less, using a large amount of solvent. However, the reaction with such a dilute solution requires large volumes, with large reactors, and substantial removal of solvent, which requires significant energy use.

Unfortunately, when a polysaccharide solution is used at a higher concentration of, for example, 10% (w/v) or more, the mixture exhibits a high viscosity and in some cases becomes non-flowing. Therefore, a strong stirring device should be used for the reaction. Even in such cases, only partial reaction may occur leading to unsatisfactory results. To reduce polysaccharide solution viscosity, there are a number of methods that can be used most commonly heating at 70° C. or more in a heterogeneous system. However, in such a method, in order to improve reaction yield, the reagent concentration is increased and the reaction time is prolonged. Furthermore, at higher temperatures, there is a possibility that a change in the chemical structure of the polysaccharide may occur, including racemization, epimerization, depolymerization of the polysaccharide or other unfavorable reactions.

Further, a method has been proposed in which a gelatinous substance including polysaccharides is subjected to ultrasonic irradiation so as to be liquefied. However, liquefaction by this method has been achieved by degrading the molecular structure of polysaccharides so as to result in depolymerization. It is not possible to use this method in the case where, for example, it is desired to perform chemical modification while maintaining control over the polysaccharide molecular weight.

### SUMMARY

Some embodiments of the present invention provides for a liquid material including a polysaccharide at high concentration with low viscosity, and its manufacturing method.

Some embodiments of the present disclosure are directed to a liquid material having a low viscosity produced by irradiating an ungelled mixture of a polysaccharide and an aqueous solvent with ultrasound. Furthermore, it has been found that liquid liquefaction can be carried out without increasing the output of the ultrasonic wave. As a result, theliquid can be liquefied without causing depolymerization of polysaccharides.

Some embodiments of the present disclosure are directed to a low viscosity liquid including a polysaccharide at a high concentration and its manufacturing method, which are described below.

Some embodiments of the present disclosure are directed to a method for producing a polysaccharide-including liquid material comprises irradiating a mixture of a polysaccharide and an aqueous solvent at a temperature lower than the gelation temperature.

In some embodiments, the concentration of the polysaccharide in the mixture is 3˜25% (w/v).

In some embodiments, the temperature at which the ultrasonic waves are applied is 30° C. or less.

In some embodiments, the frequency of the ultrasonic waves to be irradiated is 20˜400 kHz.

In some embodiments, the output of the ultrasonic waves to be irradiated is 20 W/L or less.

In some embodiments, the method for producing the polysaccharide-including liquid material is carried out under irradiation of ultrasonic waves with stirring.

In some embodiments, the method for producing a polysaccharide-including liquid material is carried out under irradiation of ultrasonic waves with bubbling of an inert gas.

Some embodiments of the present disclosure are directed to a polysaccharide-including liquid material obtained by the production processes described above.

Some embodiments of the present disclosure are directed to a liquid substance comprising a polysaccharide and an aqueous solvent, wherein the concentration of the polysaccharide in the liquid is 3-25% (w/v) and the viscosity of the liquid is 120 Pa s or less.

Some embodiments of the present disclosure are directed to a liquid substance comprising a polysaccharide and an aqueous solvent as described above in which the structure of polysaccharides is practically maintained without decomposition or degradation.

Some embodiments of the present disclosure are directed to a liquid substance comprising a polysaccharide and an aqueous solvent as described above, in which polysaccharides are selected from the group consisting of one or more compounds of amylose, amylopectin, glycogen, cellulose, chitin, chitosan, agarose, xyloglucan, glucomannan, hyaluronic acid, gellan gum, deacylated gellan gum, rhamzan gum, diutan gum, guar gum, xanthan gum, carrageenan, xanthan gum, hexuronic acid, fucoidan, pectin, pectic acid, pectinic acid, heparosan, heparan sulfate, heparin, keratan sulfate, chondroitin, chondroitin sulfate, dermatan sulfate, rhamnan sulfate, and their salts thereof.

Some embodiments of the present disclosure are directed to a pharmaceutical ingredient formed by using the polysaccharide-including liquid material as described above.

Some embodiments of the present disclosure are directed to a food ingredient formed by using the polysaccharide-including liquid material as described above.

Some embodiments of the present disclosure are directed to a nutritional supplement formed by using the polysaccharide-including liquid material as described above.

Some embodiments of the present disclosure are directed to a cosmetic or cosmeceutical ingredient formed by using the polysaccharide-including liquid material as described above.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chart of a method of producing a polysaccharide-including liquid material according to some embodiments of the present disclosure;

FIG. 2 is a schematic representation of a method of producing a polysaccharide-including liquid material according to some embodiments of the present disclosure; and

FIG. 3 is an image of a polysaccharide-including liquid material according to some embodiments of the present disclosure and an untreated liquid material.

### DETAILED DESCRIPTION

Some embodiments of the present disclosure are directed to a polysaccharide-including liquid material, having a low viscosity while the polysaccharide in the liquid substance is maintained at a high concentration without undergoing depolymerization. It can be expected to be applied to a medicine or food by utilizing the physicochemical and biological properties of polysaccharides. Also, it can be expected to contribute to the development activity of a new medicine by facilitating the chemical modification of the polysaccharide.

1. Polysaccharides

In some embodiments, polysaccharides used in production processes according to the present invention, which are not specifically restricted, include polysaccharides having 10 or more monosaccharides. In some embodiments, monosaccharide examples include but are not limited to triose, tetrose, pentose, hexose and heptose. In some embodiments, polysaccharides having one or more uronic acid or polysaccharides with part of phosphate and/or sulfate groups in the structure such as glucuronic acid, iduronic acid, galacturonic acid, mannuronic acid, and guluronic acid, can also be used. In some embodiments, natural or engineered polysaccharides produced by microorganisms or polysaccharides artificially synthesized with the use of chemical synthesis of enzymatic synthesis are also included. In some embodiments, chemical or enzymatic modification of natural or engineered polysaccharides are also included.

In some embodiments, the substance/material includes one or more monosaccharides/polysaccharides and one or more aqueous solvents. In some embodiments, the polysaccharides include one or more compounds of amylose, amylopectin, glycogen, cellulose, chitin, chitosan, agarose, xyloglucan, glucomannan, hyaluronic acid, gellan gum, deacylated gellan gum, rhamzan gum, diutan gum, guar gum, xanthan gum, carrageenan, xanthan gum, hexuronic acid, fucoidan, pectin, pectic acid, pectinic acid, heparosan, heparan sulfate, heparin, keratan sulfate, chondroitin sulfate, dermatan sulfate, rhamnan sulfate and their salts. In particular, chitin, hyaluronic acid, guar gum, xanthan gum, carrageenan, pectin, heparosan, heparin, keratan sulfate, chondroitin, chondroitin sulfate, dermatan sulfate and their salts are preferred. In some embodiments, mucopolysaccharides such as hyaluronic acid, heparin, chondroitin, chondroitin sulfate, dermatan sulfate, keratan sulfate, chitin, chitosan and their salts are more preferred, and hyaluronic acid, heparosan, and chondroitin sulfate are most preferred.

In some embodiments, the present invention, as described below, uses the condition in which mixing the polysaccharides with an aqueous solvent occurs at a temperature which does not gel. Therefore, a polysaccharide having a gelling temperature at room temperature or higher is preferable from the viewpoint of operational ease.

2. Aqueous Solvent

In some embodiments, the aqueous solvent used in the production method of the present invention includes water and/or a mixed solvent of water and an organic solvent. In some embodiments, water alone is particularly preferred. In some embodiments, when a mixed solvent of water and an organic solvent is used, the proportion of the organic solventin the mixed solvent is 10% (v/v) or less. In some embodiments , the proportion of the organic solvent in the mixed solvent is 1-5% (v/v), as long as the proportion of organic solvent in the mixture does not inhibit dissolution of the polysaccharide. The aqueous solvent or aqueous cosolvent with organic solvents to be used may be selected in accordance with the use of the resulting polysaccharide-including solution.

In some embodiments, the organic solvent used for the mixed solvent of water and an organic solvent are not specifically restricted if it can mix with water, including ketones such as acetone and methyl ethyl ketone, alcohols such as methanol and ethanol, ethers such as tetrahydrofuran, dioxane and ethylene glycol dimethyl ether, amides such as dimethylformamide, diethylformamide and dimethylacetamide, sulfoxides such as dimethyl sulfoxide and diethyl sulfoxide, amines such as pyridine, nitriles such as acetonitrile, carboxylic acids such as formic acid and acetic acid, hexamethylphosphoric triamide (HMPA), and so on, or combinations thereof.

3. Manufacturing

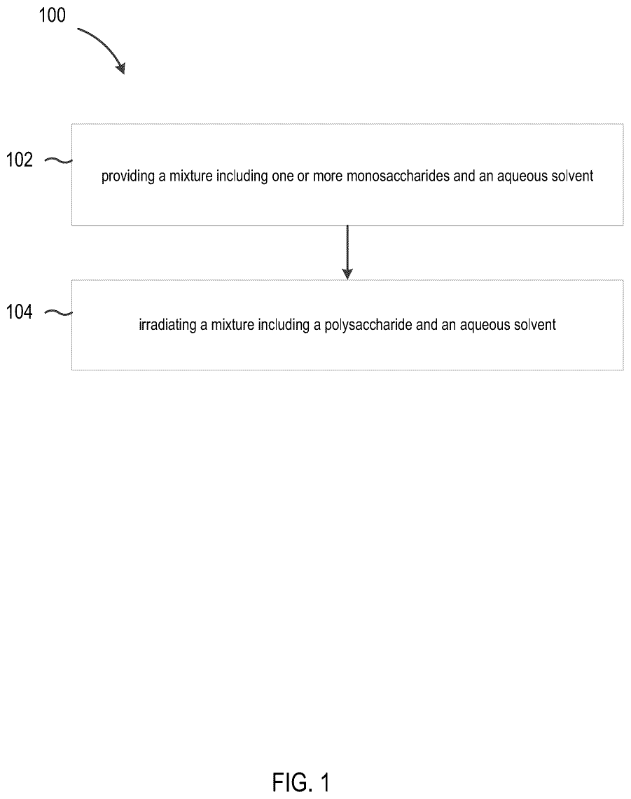

Referring now to FIG. 1, some aspects of the present disclosure include a method 100 for producing a polysaccharide-including liquid material. At 102, a mixture including one or more monosaccharides and an aqueous solvent is provided. In some embodiments, the one or more monosaccharides includes a polysaccharide. At 104, the mixture including a polysaccharide and an aqueous solvent is irradiated. As discussed above, in some embodiments, the polysaccharide includes one or more uronic acid, polysaccharides with part of phosphate and/or sulfate groups in the structure such as glucuronic acid, iduronic acid, galacturonic acid, mannuronic acid, and guluronic acid, natural or engineered polysaccharides produced by microorganisms or polysaccharides artificially synthesized with the use of chemical synthesis of enzymatic synthesis, chemical or enzymatic modified natural or engineered polysaccharides, etc., or combinations thereof. Also as discussed above, in some embodiments, the aqueous solvent includes water and/or a mixed solvent of water and an organic solvent. In some embodiments, when a mixed solvent of water and an organic solvent is used, the proportion of the organic solventin the mixed solvent is 10% (v/v) or less. In some embodiments, the proportion of the organic solvent in the mixed solvent is 1-5% (v/v), as long as the proportion of organic solvent in the mixture does not inhibit dissolution of the polysaccharide. In some embodiments, the organic solvent includes ketones such as acetone and methyl ethyl ketone, alcohols such as methanol and ethanol, ethers such as tetrahydrofuran, dioxane and ethylene glycol dimethyl ether, amides such as dimethylformamide, diethylformamide and dimethylacetamide, sulfoxides such as dimethyl sulfoxide and diethyl sulfoxide, amines such as pyridine, nitriles such as acetonitrile, carboxylic acids such as formic acid and acetic acid, hexamethylphosphoric triamide (HMPA), and so on, or combinations thereof.

In some embodiments, the concentration of the polysaccharide in the mixture is between about 3% and about 25% (w/v). In some embodiments, the concentration of the polysaccharide in the mixture is between about 8% and about 18% (w/v).

In some embodiments, irradiating step 102 occurs at a temperature lower than the gelation temperature of the mixture. In some embodiments, the mixture is irradiated with ultrasonic waves. In some embodiments, the mixture is irradiated with ultrasonic waves via any suitable process known to those of skill in the art, e.g., via a probe sonicator, etc. In some embodiments, the frequency of the ultrasonic waves is between about 20 kHz and about 400 kHz. In some embodiments, the output of the ultrasonic waves is about 20 W/L or less. In some embodiments, the output of the ultrasonic waves is applied to the mixture for between about 5 minutes and about 30 minutes. In some embodiments, the temperature at which the ultrasonic waves are applied is between about 1° C. and about 25° C. below a gelation temperature of the mixture. In some embodiments, the temperature at which the ultrasonic waves are applied is less than about 30° C. As discussed above, in some embodiments, the material includes amylose, amylopectin, glycogen, cellulose, chitin, chitosan, agarose, xyloglucan, glucomannan, hyaluronic acid, gellan gum, deacylated gellan gum, rhamzan gum, diutan gum, guar gum, xanthan gum, carrageenan, xanthan gum, hexuronic acid, fucoidan, pectin, pectic acid, pectinic acid, heparosan, heparan sulfate, heparin, keratan sulfate, chondroitin sulfate, dermatan sulfate, rhamnan sulfate, their salts, or combinations thereof.

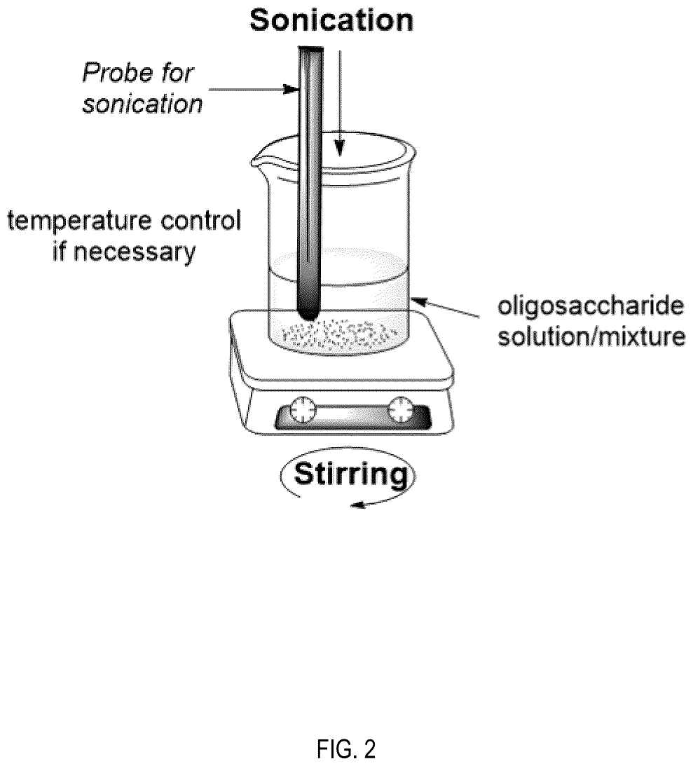

Referring now to FIG. 2, as discussed above, in some embodiments, the method for manufacturing a liquid material according to the present invention applies ultrasound to a mixture of polysaccharides and an aqueous solvent or an aqueous cosolvent with organic solvents at a temperature, which does not cause gelation.

In some embodiments, the mixture of the polysaccharide and the aqueous solvent is prepared by mixing at a temperature at which gelation does not occur. If ultrasound is applied to a gelled mixture, a liquid material with a low viscosity and without depolymerization cannot be obtained.

The ratio of the polysaccharide in the mixture of the polysaccharide and the aqueous solvent is usually 3 to 25% (w/v), preferably 5 to 20% (w/v), more preferably 8 to 18% (w/v).

The temperature at which gelation does not occur varies depending on the type and concentration of polysaccharide used, the type of aqueous solvent, etc., and therefore should be set as appropriate. In some embodiments, the temperature is set to about 1° C. to about 25° C. lower than the gelation temperature ofpolysaccharide used. In some embodiments, the temperature is set to between 5-20° C. lower than the gelation temperature of polysaccharide used. In some embodiments, the temperature is set to about 8° C. to about 15° C. lower than the gelation temperature of polysaccharide used. In some embodiments, the temperature is set to about 15° C. lower than the gelation temperature of polysaccharide used. In some embodiments, the temperature is set to about 10° C. lower than the gelation temperature of polysaccharide used. For example, in the case of a 10% (w/v) hyaluronic acid/water mixture, gelation occurs at room temperature (around 25° C.). In some embodiments, the temperature would be set to between 5-20° C. In some embodiments, the temperature would be set to about 15° C.

In some embodiments, the mixture of the polysaccharide and the aqueous solvent includes a heparosan/water mixture, a chondroitin sulfate/water mixture, a pectin/water mixture, a carrageenan/water mixture, a guar gum/water mixture, a xanthan gum/water mixture, or combinations thereof.

By way of example, in the case of a 10% (w/v) heparosan/water mixture, it is preferable to set the temperature between 5-20° C., more preferably around 15° C. In the case of a 10% (w/v) chondroitin sulfate/water mixture, it is preferable to set the temperature between 5-20° C. more preferably around 15° C. In the case of a 10% (w/v) pectin/water mixture, it is preferable to set the temperature between 5-20° C., more preferably around 15° C. In the case of a 10% (w/v) carrageenan/water mixture, it is preferable to set the temperature between 5-20° C., more preferably around 15° C. In the case of a 10% (w/v) guar gum/water mixture, it is preferable to set the temperature between 5-20° C., more preferably around 15° C. In the case of a 10% (w/v) xanthan gum/water mixture, it is preferable to set the temperature between 5-20° C., more preferably around 15° C.

From the viewpoint of achieving low viscosity and suppressing polysaccharide depolymerization, which are the characteristics of the liquid material of the present invention, the irradiation time, frequency, and output of the ultrasonic wave are set as follows.

In some embodiments, the irradiation time of the ultrasonic wave applied to the mixture of the polysaccharide and the aqueous solvent range from about 0.1 second to about 60 min. In some embodiments, the irradiation time of the ultrasonic wave applied to the mixture of the polysaccharide and the aqueous solvent range from preferably about 1 to about 40 min. In some embodiments, the irradiation time of the ultrasonic wave applied to the mixture of the polysaccharide and the aqueous solvent range from about 5 to about 30 min.

In some embodiments, the frequency of ultrasonic waves applied to the mixture of polysaccharide and aqueous solvent is between about 20 kHz and about 400 kHz. In some embodiments, the frequency of ultrasonic waves applied to the mixture of polysaccharide and aqueous solvent is between about 10 kHz and about 100 kHz. In some embodiments, the frequency of ultrasonic waves applied to the mixture of polysaccharide and aqueous solvent is between about 20 kHz and about 60 kHz. In some embodiments, the frequency of ultrasonic waves applied to the mixture of polysaccharide and aqueous solvent is between about 30 kHz and about 50 kHz.

In some embodiments, the output of the ultrasonic wave applied to the mixture of polysaccharide and aqueous solvent is about 1 to about 50 W/L. In some embodiments, the output of the ultrasonic wave applied to the mixture of polysaccharide and aqueous solvent is about 2 to about 30 W/L. In some embodiments, the output of the ultrasonic wave applied to the mixture of polysaccharide and aqueous solvent is about 5 to about 20 W/L. In some embodiments, the output of the ultrasonic waves is about 20 W/L or less.

In some embodiments, the temperature at which the ultrasonic waves are applied is between about 0 to about 20° C. In some embodiments, the temperature at which the ultrasonic waves are applied is between about 10 to about 15° C.

In some embodiments, ultrasonic irradiation is performed to the mixture of polysaccharide and aqueous solvent while stirring. The stirring is not particularly limited and may be a method using a high-speed rotary shearing device such as a mechanical stirrer or a magnetic stirrer, a bubbling method for introducing an inert gas into the system, and the like. A method using these methods in combination may also be used. In some embodiments, as the inert gas used in the bubbling method, a gas inert to polysaccharides such as nitrogen gas, helium gas and argon gas may be used, and nitrogen gas is preferable from an economical viewpoint. In some embodiments, from the viewpoint of stirring efficiency, the inert gas is preferably introduced from below the mixture. The conditions of use of the inert gas are not particularly limited, and may be used by appropriately adjusting the volume of the mixture, the temperature and the length of the introduction tube, under consideration of the mixture volume, temperature so that the stirring is sufficiently performed under ultrasound.

4. Polysaccharide-Including Liquid Material

In some embodiments, the polysaccharide-including liquid material of the present invention (hereinafter sometimes referred to as the present liquid material) is a liquid material including a polysaccharide and a solvent. As discussed above, in some embodiments, the solvent can be either an aqueous solvent or an aqueous cosolvent with organic solvents. The present liquid material includes polysaccharides at a high concentration and has low viscosity.

In some embodiments, the concentration of the polysaccharide in the liquid is 3 to 25% (w/v), preferably 5 to 20% (w/v), and more preferably 8 to 18% (w/v).

The viscosity of the liquid material varies depending on the type and concentration of the polysaccharide to be used. In some embodiments, the viscosity is about 120 Pa s or less, such as, 1 to 120 Pa s, 5 to 118 Pa s, 8 to 100 Pa s, and the like. This viscosity is determined on a measured value at 25° C., using a B-type viscometer (manufactured by Toki Sangyo Co., Ltd., model number TV-22). Moreover, in the polysaccharide of the present liquid material, the depolymerization (decomposition, disassembly) of polysaccharide does not occur to any substantial degree, and the molecular structure of the polysaccharide is maintained. Here, “substantial” means that even if depolymerization of the polysaccharide occurs, the depolymerization degree is acceptable to the present liquid material. In some embodiments, when measuring the Mₙ of the polysaccharide, the ratio between the Mₙ of raw material and the Mₙ of ultrasound resulted material should be at least 90% or more, preferably 95% or more, and more preferably 98% or more.

More surprisingly, the liquid material does not gel even when the temperature is raised, and thus, its fluidity is maintained.

The liquid material is excellent in fluidity despite polysaccharides being maintained at a high concentration with the molecular structure of the polysaccharide maintained in the liquid material. Therefore, it is not only applicable to pharmaceuticals and health foods that use the properties of polysaccharides as they are, but also has excellent stirring efficiency due to its fluidity. Further, the chemical modification of polysaccharides, for example, hydrolysis of the acetamide group of mucopolysaccharides and introduction of hydroxyl protecting groups, can be efficiently performed. Finally, compared with conventional reactions with a low concentration solution, the amount of products that can be produced in a given amount of time is significantly increased. It is possible to contribute to the reduction of the amount of solvent used and the energy required for the reaction and purification.

### EXAMPLES

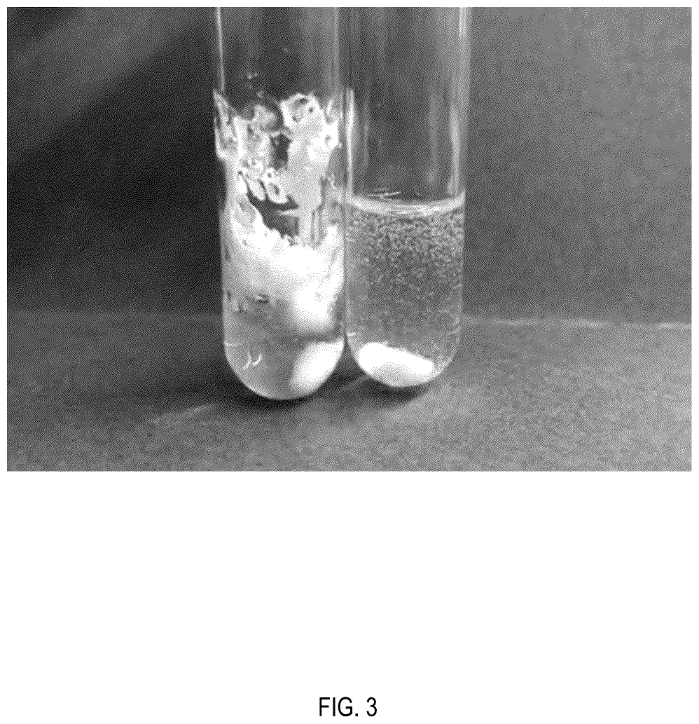

Hereinafter, the present invention will be specifically described with reference to Examples and Comparative Examples, such as the treated and untreated solutions shown in FIG. 3. However, the examples are merely examples, and the present invention is not limited to the examples.

For ultrasonic irradiation in the following examples and comparative examples, a Bransonic tabletop ultrasonic cleaner 5510 (manufactured by Nippon Emerson; frequency 42 kHz, output 18.9 W/L) was used.

The viscosity (at 25° C.) of the polysaccharide-including liquids obtained in the following examples and comparative examples was measured using an E-type viscometer (manufactured by Toki Sangyo Co., Ltd., model number TV-22).

Moreover, the number average molecular weight (Me) of the polysaccharide in the liquid material used or obtained in the following Examples and Comparative Examples is measured through a gel permeation chromatograph “Prominence” (GPC) manufactured by Shimadzu Corporation, column used: TSKgel guard column PWXL+TSKgelG3000PWXL+TSKgel G4000PWXL, solvent: ammonium acetate+sodium azide aqueous solution, reference sample: TSKgel standard polyethylene oxide, measured temperature: 30° C.

### Example 1

Sodium hyaluronate (number average molecular weight 50,000 to 110,000; manufactured by Kikkoman Biochemifa Co., Ltd., the same shall apply hereinafter) 0.5 g in terms of solid content was charged into 5.0 g of deionized ultrapure water (15° C.). At this point, sodium hyaluronate was precipitated without dissolving in water. While maintaining the temperature, ultrasonic irradiation was performed for 10 min while gently bubbling with nitrogen gas. Ultrasonic irradiation and bubbling were then stopped, and the temperature was gradually raised to 25° C. with stirring. It was confirmed that a transparent liquid material was obtained.

The viscosity of the obtained liquid was 16 Pa s, and it was easily stirred.

Further, the Mₙ of the polysaccharide in the resulting liquid was measured and no decrease in molecular weight was observed, confirming that no depolymerization occurred.

### Comparative Example 1

Sodium hyaluronate 0.5 g in terms of solid content was charged into 5.0 g of deionized ultrapure water, heated to 80° C. and then stirred for 1 h to obtain a solution with a relatively low viscosity. When this solution was cooled down to 15° C., it completely solidified. Ultrasonic irradiation was performed for 10 min while gently bubbling with nitrogen gas at 25° C. to obtain a pale yellow liquid.

The viscosity of the obtained liquid material was 155 Pa s, and it was a low fluidity material that was difficult to stir.

### Comparative Example 2

Sodium hyaluronate 0.5 g in terms of solid content was charged into 5.0 g of deionized ultrapure water (15° C.) with stirring. After maintaining the temperature for 10 min after charging, the temperature was gradually raised to 25° C.

The viscosity of the obtained mixture was 161 Pa s, and it was a low fluidity material that was difficult to stir.

### Example 2

Sodium hyaluronate 0.75 g in terms of solid content was charged into 5.0 g of deionized ultrapure water (15° C.). At this point, sodium hyaluronate was precipitatedwithout dissolving in water. While maintaining the temperature, ultrasonic irradiation was performed for 10 min while gently bubbling with nitrogen gas. Slight insoluble matter was observed, therefore the process was continued for another 10 min (20 min in total). Then, ultrasonic irradiation and bubbling were stopped, and the temperature was gradually raised to 25° C. with stirring. It was confirmed that a transparent liquid material was obtained.

The viscosity of the obtained liquid was 26 Pa s, and it was easily stirred.

Further, the Mₙ of the polysaccharide in the obtained liquid was measured and no decrease in molecular weight was observed, confirming that no depolymerization occurred.

### Comparative Example 3

Sodium hyaluronate 0.75 g in terms of solid content was charged into 5.0 g of deionized ultrapure water (15° C.) with stirring. After maintaining the temperature for 10 min after charging, the temperature was gradually raised to 25° C.

The viscosity of the obtained mixture was 398 Pa s, and it was a non-liquid material that was difficult to stir.

### Example 3

Sodium hyaluronate 1.0 g in terms of solid content was charged into 5.0 g of deionized ultrapure water (15° C.). At this point, sodium hyaluronate was precipitatedwithout dissolving in water. While maintaining the temperature, ultrasonic irradiation was performed for 10 min while gently bubbling with nitrogen gas. Slight insoluble matter was observed, therefore the process was continued for another 10 min (20 min in total). Then, ultrasonic irradiation and bubbling were stopped, and the temperature was gradually raised to 25° C. with stirring. It was confirmed that a transparent liquid material was obtained.

The viscosity of the obtained liquid was 116 Pa s, and it was easily stirred.

Further, the Mₙ of the polysaccharide in the obtained liquid was measured and no decrease in molecular weight was observed, confirming that no depolymerization occurred.

### Comparative Example 4

Sodium hyaluronate 1.0 g in terms of solid content was charged into 5.0 g of deionized ultrapure water (15° C.) with stirring. After maintaining the temperature for 10 min after charging, the temperature was gradually raised to 25° C.

The viscosity of the obtained mixture was 453 Pa s, and it was a non-liquid material that was difficult to stir.

### Reference Example

### Deacetylation of Sodium Hyaluronate

The liquid obtained in Example 1 was charged with 2.5 mL of an 8N aqueous sodium hydroxide solution and stirred for 16 h. Then, 10 mL of ethanol was added to the reaction solution, and the deposited precipitate was collected by filtration and dried under reduced pressure to obtain a white solid.

Based the ¹H-NMR spectrum analysis of the obtained white solid, it was confirmed that the deacetylation reaction had completely occurred.

### Example 4

Heparosan (molecular weight 50,000 to 120,000; obtained from Sigma-Aldrich Japan GK, the same shall apply hereinafter) 0.5 g in terms of solid content was charged into 5.0 g of deionized ultrapure water (15° C.). Ultrasonic irradiation was performed for 10 min while gently bubbling with nitrogen gas. Bubbling was stopped and the temperature was gradually raised to 25° C. with stirring to obtain a yellow translucent liquid.

The viscosity of the obtained liquid was 16 Pa s, and it was easily stirred.

Further, when the Mₙ of the polysaccharide in the obtained liquid was measured, no decrease in molecular weight was observed, and it was confirmed that no depolymerization occurred.

On the other hand, in the case when 0.5 g of heparosan was put into 5.0 g of deionized ultrapure water and directly stirred at 25° C., the solid was still precipitated in a powder form.

### Example 5

Chondroitin sulfate A sodium (Molecular weight 50,000-100,000; obtained from Sigma-Aldrich Japan GK) 0.5 g in terms of solid content was charged into 5.0 g of deionized ultrapure water (15° C.), and ultrasonic irradiation was performed for 10 min while gently bubbling with nitrogen gas. Then ultrasonic irradiation and bubbling were stopped, and the temperature was gradually raised to 25° C. with stirring to obtain a transparent liquid.

The viscosity of the obtained liquid was 16 Pa s, and it was easily stirred.

On the other hand, 0.5 g of chondroitin sulfate A sodium was put into 5.0 g of deionized ultrapure water and stirred at 25° C., the solid was still precipitated in a powder form.

### Example 6

Pectin (Molecular weight 50,000-360,000; obtained from Sigma-Aldrich Japan GK) 0.5 g in terms of solid content was charged into 5.0 g of deionized ultrapure water (15° C.), and ultrasonic irradiation was performed for 10 min while gently bubbling with nitrogen gas. Then ultrasonic irradiation and bubbling were stopped, and the temperature was gradually raised to 25° C. with stirring to obtain a transparent liquid.

The viscosity of the obtained liquid was 12 Pa s, and it was easily stirred.

On the other hand, 0.5 g of Pectin was put into 5.0 g of deionized ultrapure water and stirred at 25° C., the solid was still precipitated in a powder form.

### Example 7

Carrageenan (Molecular weight 100,000-150,000; obtained from Sigma-Aldrich Japan GK) 0.5 g in terms of solid content was charged into 5.0 g of deionized ultrapure water (15° C.), and ultrasonic irradiation was performed for 10 min while gently bubbling with nitrogen gas. Then ultrasonic irradiation and bubbling were stopped, and the temperature was gradually raised to 25° C. with stirring to obtain a creamy translucent liquid.

The viscosity of the obtained liquid was 13 Pa s, and it was easily stirred.

On the other hand, 0.5 g of Carrageenan was put into 5.0 g of deionized ultrapure water and stirred at 25° C., the solid was still precipitated in a powder form.

### Example 8

Guar gum (Molecular weight 200,000-300,000; obtained from Sigma Aldrich Japan GK) 0.5 g in terms of solid content was charged into 5.0 g of deionized ultrapure water (15° C.), and ultrasonic irradiation was performed for 10 min while gently bubbling with nitrogen gas. Then ultrasonic irradiation and bubbling were stopped, and the temperature was gradually raised to 25° C. with stirring to obtain a brownish translucent liquid.

The viscosity of the obtained liquid was 12 Pa s, and it was easily stirred.

On the other hand, 0.5 g of Guar gum was put into 5.0 g of deionized ultrapure water and stirred at 25° C., the solid was still precipitated in a powder form.

### Example 9

Xanthan gum (Molecular weight 2,000,000; obtained from by Sigma-Aldrich Japan GK) 0.5 g in terms of solid content was charged into 5.0 g of deionized ultrapure water (15° C.), and ultrasonic irradiation was performed for 10 min while gently bubbling with nitrogen gas. Then ultrasonic irradiation and bubbling were stopped, and the temperature was gradually raised to 25° C. with stirring to obtain a light-yellow translucent liquid.

The viscosity of the obtained liquid was 18 Pa s, and it was easily stirred.

On the other hand, 0.5 g of Xanthan gum was put into 5.0 g of deionized ultrapure water and stirred at 25° C., the solid was still precipitated in a powder form.

### INDUSTRIAL APPLICABILITY

By using the production method of the present invention, a liquid substance with a low viscosity can be provided while maintaining a polysaccharide at a high concentration. Since the polysaccharide in the liquid substance does not undergo depolymerization, the polysaccharide can exhibit its inherent properties, and can be used for chemical modification.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method for producing a polysaccharide-including liquid material, comprising:
irradiating a mixture including a polysaccharide and an aqueous solvent,
wherein the irradiating step occurs at a temperature lower than the gelation temperature of the mixture.

2. The method according to claim 1, wherein the concentration of the polysaccharide in the mixture is between about 3% and about 25% (w/v).

3. The method according to claim 2, wherein the concentration of the polysaccharide in the mixture is between about 8% and about 18% (w/v).

4. The method according to claim 1, wherein the mixture is irradiated with ultrasonic waves. The method according to claim 4, wherein the temperature at which the ultrasonic waves are applied is between about 1° C. and about 25° C. below a gelation temperature of the mixture.

6. The method according to claim 4, wherein the temperature at which the ultrasonic waves are applied is less than about 30° C.

7. The method according to claim 4, wherein the frequency of the ultrasonic waves is between about 20 kHz and about 400 kHz.

8. The method according to claim 4, wherein the output of the ultrasonic waves is about 20 W/L or less.

9. The method according to claim 4, wherein the output of the ultrasonic waves is applied to the mixture for between about 5 minutes and about 30 minutes. The method according to claim 4, wherein irradiating the mixture including a polysaccharide and an aqueous solvent further comprises stirring the mixture.

11. The method according to claim 10, wherein stirring the mixture further comprises bubbling of an inert gas through the mixture.

12. The method according to claim 1, wherein the material includes amylose, amylopectin, glycogen, cellulose, chitin, chitosan, agarose, xyloglucan, glucomannan, hyaluronic acid, gellan gum, deacylated gellan gum, rhamzan gum, diutan gum, guar gum, xanthan gum, carrageenan, xanthan gum, hexuronic acid, fucoidan, pectin, pectic acid, pectinic acid, heparosan, heparan sulfate, heparin, keratan sulfate, chondroitin sulfate, dermatan sulfate, rhamnan sulfate, their salts, or combinations thereof.

13. The method according to claim 1, wherein the aqueous solvent includes less than about 10% (v/v) organic solvent.

14. A polysaccharide-including liquid material obtained by the method according to claim 1, wherein the concentration of the polysaccharide in the liquid is 3-25% (w/v) and the viscosity of the liquid is about 120 Pa s or less.

15. The liquid material according to claim 14, wherein the structure of polysaccharides are practically maintained without decomposition or degradation.

16. The liquid material according to claim 14, wherein the polysaccharides includes amylose, amylopectin, glycogen, cellulose, chitin, chitosan, agarose, xyloglucan, glucomannan, hyaluronic acid, gellan gum, deacylated gellan gum, rhamzan gum, diutan gum, guar gum, xanthan gum, carrageenan, xanthan gum, hexuronic acid, fucoidan, pectin, pectic acid, pectinic acid, heparosan, heparan sulfate, heparin, keratan sulfate, chondroitin sulfate, dermatan sulfate, rhamnan sulfate, their salts, or combinations thereof.

17. The liquid material according to claim 14, wherein the mixture is irradiated with ultrasonic waves.

18. The liquid material according to claim 17, wherein the temperature at which the ultrasonic waves are applied is between about 1° C. and about 25° C. below a gelation temperature of the mixture.

19. The liquid material according to claim 17, wherein the mixture is stirred via bubbling of an inert gas through the mixture. A pharmaceutical ingredient including the polysaccharide-including liquid material according to claim 14.

21. A food ingredient including the polysaccharide-including liquid material according to claim 14.

22. A nutritional supplement including the polysaccharide-including liquid material according to claim 14.

23. A cosmetic or cosmeceutical ingredient including the polysaccharide-including liquid material according to claim 14.
